(19) United States
(12) Patent Application Publication
(10) Pub. No.: US 2023/0190620 A1
(43) Pub. Date: Jun. 22, 2023

(54) MOISTURIZER COMPOSITIONS AND METHODS OF MAKING THE SAME

(71) Applicant: Zahra MOTAHARI, Laguna Hills, CA (US)

(72) Inventor: Zahra MOTAHARI, Laguna Hills, CA (US)

(21) Appl. No.: 18/083,133

(22) Filed: Dec. 16, 2022

Related U.S. Application Data
(60) Provisional application No. 63/290,335, filed on Dec. 16, 2021.

(51) Int. Cl. A61K 8/67; A61K 8/49; A61K 8/362; A61K 8/19; A61K 8/92; A61Q 19/00

(52) U.S. Cl. CPC A61K 8/675; A61K 8/673; A61K 8/4926; A61K 8/362; A61K 8/676; A61K 8/19; A61K 8/922; A61Q 19/007; A61K 2800/524; A61K 2800/48

(57) ABSTRACT

Moisturizer compositions include about 1 to about 5 wt % niacinamide, about 0.5 to about 2 wt % pantothenic acid, about 0.5 to about 2 wt % pyridoxine, about 0.05 to about 1 wt % of a skin barrier enhancer, about 1 to about 10 wt % of a humectant, about 1 to about 10 wt % citric acid, about 0.1 to about 0.5 wt % an ascorbic acid salt or an ascorbic acid derivative, about 0.01 to about 0.2 wt % of a non-naturally occurring preservative, about 0.5 to about 2 wt % of a natural preservative and balance of a water:glycerin mixture, where the water:glycerin mixture has at least about 50 wt % water.

### CROSS-REFERENCE TO RELATED APPLICATION

This application claims the benefit of U.S. Provisional Application No. 63/290,335 filed Dec. 16, 2021, the entire contents of which are incorporated by reference herein.

### FIELD OF THE INVENTION

The present invention relates to moisturizer compositions, methods of making said moisturizer compositions, and methods of using said moisturizer compositions. More specifically, the present invention relates to moisturizer compositions for topical application onto the skin of a mammal.

### BACKGROUND OF THE DISCLOSURE

Moisturizers are designed to prevent and treat dry skin. They can also protect sensitive skin, improve skin texture and mask imperfections. Individual components of a moisturizer may have different effects on a user, so one may need to try various products before the right moisturizer is found. The moisturizer that works best for an individual may depend on a variety of factors, including skin type (for example, normal, dry oily, sensitive), age and the presence specific conditions, such as acne.

Moisturizers are generally one of oil-based, water-based, or occlusive. Moisturizers may be in the form of gels, lotions creams, ointments, or mists/sprays. Oil-based moisturizers are generally classified as emollients. Emollients usually contain oil and lipid (fatty acid) ingredients such as ceramides, cocoa butter, collagen, dimethicone, lanolin, mineral oil, shea butter, or jojoba oil. These ingredients improve the texture and appearance of the skin by filling in its crevices to make it look smoother. Certain emollients, such as collagen, are used for their anti-aging properties and can reduce the appearance of fine lines and wrinkles. Other emollients like ceramides can help treat eczema. Water-based moisturizers are generally classified as humectants. Humectants usually contain ingredients such as ingredients such as hyaluronic acid, glycerin, lactic acid, alpha hydroxy acids (AHA), or urea. They hydrate the skin by drawing moisture from the air to the outer layers of the skin, the epidermis, and stratum corneum, replenishing the skin. Occlusive moisturizers usually contain oil ingredients, such as beeswax, petrolatum, silicones, and petroleum jelly, that are intended to provide a protective seal over the skin to prevent moisture loss.

### SUMMARY OF THE INVENTION

The present invention relates to moisturizer compositions, methods of making said moisturizer compositions, and methods of using said moisturizer compositions.

A first embodiment of the invention is a moisturizer composition comprising about 1 to about 5 wt % niacinamide (Vitamin B3), about 0.5 to about 2 wt % pantothenic acid (Vitamin B5), about 0.5 to about 2 wt % pyridoxine (Vitamin B6), about 0.05 to about 1 wt % of a skin barrier enhancer, about 1 to about 10 wt % of a humectant, about 1 to about 10 wt % citric acid, about 0.1 to about 0.5 wt % of an ascorbic acid salt or an ascorbic acid derivative, about 0.01 to about 0.2 wt % of a non-naturally occurring preservative, about 0.5 to about 2 wt % of a natural preservative, with the balance of the moisturizer composition being a water:glycerin mixture, where the water:glycerin mixture has at least about 50 wt % water.

A second embodiment of the invention is a moisturizer composition according to the first embodiment, wherein the water:glycerin mixture has a water:glycerin weight ratio ranging from about 50:50 to about 80:20.

A third embodiment of the invention is a moisturizer composition according to the first embodiment, wherein the water:glycerin mixture has a water:glycerin weight ratio ranging from about 50:50 to about 70:30.

A fourth embodiment of the invention is a moisturizer composition according to the first embodiment, wherein the water:glycerin mixture has a water:glycerin weight ratio ranging from about 50:50 to about 60:40.

A fifth embodiment of the invention is a moisturizer composition according to any one of the first through fourth embodiments, wherein the skin barrier enhancer is selected from the group consisting of potassium phosphate, dipotassium phosphate, phosphoric acid, squalane, squalene, linoleic acid, jojoba oil, coconut oil, almond oil, argan oil, borage oil, rosehip oil, sunflower oil, soybean oil, oat oil, palm kernel oil, and ceramides.

A sixth embodiment of the invention is a moisturizer composition according to any one of the first through fifth embodiments, wherein the humectant is selected from the group consisting of urea, sorbitol, mannitol, propylene glycol, beet root extract, Aloe barbadensis leaf extract, Aloe andongensis leaf juice, Aloe andongensis extract, Albatrellus conjluens extract, aluminum dihydroxy allantoinate (allantoin), Averrhoa carambola leaf extract, Backhousia anisata leaf extract, Banksia spinulosa flower extract, Bidens pilosa extract, Ceramium rubrum extract, Citrus auratium Tachibana peel extract, Cnidium monnieri fruit extract, Cocos nucifera fruit juice, Coleus scutellarioides extract, Convolvulus arvensis extract, Dunaliella bardawill extract, Echium lycosis root extract, Entada phaseoloides leaf extract, Erica canaliculate extract, fig fruit extract, Ganoderma atrum extract, Ganoderma neo-japonicum extract, Gentiana prostrate extract, licorice root extract, Legenaria siceraria fruit extract, Lantana camara root extract, Larix europaea wood extract, Lepidium meyenii root extract, Lespedeza bicolor bark extract, Lycopodium clavatum extract, Macrotomia euchroma root extract, Magnolia kobus bark extract, Momordica charantia fruit extract, Momordica grosvenori fruit juice, Paulownia tomentosa leaf extract, Phalaenopsis amabillis extract, Phaseolus trilobus seed extract, Portulaca grandiflora extract, Portulaca oleracea extract, Primu glycolicla sikkimensis flower extract, Prostanthera incise leaf extract, Prunus persica bud extract, Pueraria lobata root extract, Ribes nigrum seed extract, Rosa rugosa leaf extract, Saussurea involucrata extract, Scutellaria baicalensis root extract, Sesamum indicum sprout extract, jojoba leaf extract, honey extract, albumen extract, algae oligosaccharides, agarose, lactose, jellyfish extract, fructose, fructan, diglycerin, glyceryl triacetate (triacetin), glucose, acetamide MEA, acetyl histidine, anserine, calcium fructoborate, cellulose succinate, choline chloride, erythritol, ethyl glucoside, 1,3-butylene glycol, hexylene glycol, hydrolyzed corn starch, hydroxylated jojoba oil, lactic acid, sodium lactate, lysine PCA, polyglucuronic acid, sodium PCA, maltose, mannose, maltitol, melibiose, dimethyl imidazolidinone, sodium hyaluronate, hyaluronic acid, salicylic acid, glycolic acid, phytic acid, gluconolactone, xylitol, xylose, and any combination thereof.

A seventh embodiment of the invention is a moisturizer composition according to any one of the first through sixth embodiments, wherein the humectant is urea.

An eighth embodiment of the invention is a moisturizer composition according to any one of the first through seventh embodiments, wherein the non-naturally occurring preservative is selected from the group consisting of potassium sorbate, triclosan, methylisothiazolinone, chlorphenesin, chloroxylenol, iodopropynyl butyl carbamate, methyldibromo glutaronitrile, formaldehyde, benzylhemiformal, diasolidinyl urea, imidazolidinyl urea, 2-bromo-2-nitropropane-1,3-diol, 1,3-bis(hydroxymethyl)-5,5-dimethylimidazolidine-2,4-dione, 1-(hydroxymethyl)-5,5-dimethylhydantoin, 1-(3-chloroallyl)-3,5,7-triaza-1-azoniaadamantane chloride, Sodium hydroxymethyl glycinate, phenoxyethanol, 2-butoxyethanol, 2-(2-butoxyethoxy)-ethanol, 2-(2-ethoxy)-ethanol, methylparaben, ethylparaben, propylparaben, butylparaben, isobutylparaben, 1,2-hexanediol, caprylyl glycol, caprylyl glyceryl ether, dehydroacetic acid, caprylhydroxamic acid, ethylhexylglycerin, glyceryl undecylenate, glycerol, piroctone olamine, and any combination thereof.

A ninth embodiment of the invention is a moisturizer composition according to any one of the first through eighth embodiments, wherein the non-naturally occurring preservative is potassium sorbate.

A tenth embodiment of the invention is a moisturizer composition according to any one of the first through ninth embodiments, wherein the natural preservative is selected from the group consisting of benzoic acid, benzyl alcohol, sodium benzoate, potassium benzoate, sorbic acid, salicylic acid, p-anisic acid, Lactobacillus ferment, Lactobacillus, Cocos Nucifera (coconut) fruit extract, Japanese honeysuckle extract, Leuconostoc/radish root ferment filtrate, Populus Tremuloides bark extract, Salix Nigra (willow) bark extract, Leuconostoc ferment filtrate, Sambucus Nigra fruit extract, Musa Sapientum (banana) fruit extract, sodium levulinate, sodium anisate, glycerin, citrus reticulata fruit extract, citrus aurantium amara fruit extract, Citrus sinensis peel extract, ascorbic acid, citric acid, lactic acid, D-glucono-1,5-lactone, D-gluconic acid calcium salt, pentylene glycol, phenethyl alcohol, Naticide and any combination thereof.

An eleventh embodiment of the invention is a moisturizer composition according to any one of the first through tenth embodiments, wherein the natural preservative is Naticide.

A twelfth embodiment of the invention is a moisturizer composition according to any one of the first through eleventh embodiments, wherein the ascorbic acid salt is calcium L-ascorbate.

A thirteenth embodiment of the invention is a moisturizer composition according to any one of the first through eleventh embodiments, wherein the moisturizer composition further comprises about 0.5 to about 2 wt % of an essential oil.

A fourteenth embodiment of the invention is a moisturizer composition according to the thirteenth embodiment, wherein the essential oil is selected from the group consisting of almond oil, arborvitae oil, argan oil, avocado oil, babassu oil, basil oil, bergamot oil, camellia seed oil, canola oil, carrot seed oil, cassia oil, castor oil, cedarwood oil, cinnamon oil, clary sage oil, clove oil, coconut oil, copaiba oil, eucalyptus oil, frankincense oil, geranium oil, grapefruit oil, grapeseed oil, helichrysum oil, hyssop oil, jojoba oil, lavender oil, lemon oil, lemongrass oil, manila oil, moringa oil, myrrh oil, neroli oil, olive oil, orange oil, oregano oil, palmarosa oil, patchouli oil, peppermint oil, pine oil, pomegranate oil, roman chamomile oil, rice bran oil, rose oil, rosehip oil, rosemary oil, sandalwood oil, spearmint oil, sunflower oil, sweet orange oil, tangerine oil, tea tree oil, thyme oil, wintergreen oil, vetiver oil, ylang oil, and any combination thereof.

A fifteenth embodiment of the invention is a moisturizer composition according to the first embodiment, wherein the composition comprises about 2.5 wt % niacinamide, about 1.2 wt % pantothenic acid, about 1.2 wt % pyridoxine, about 0.2 wt % of the skin barrier enhancer, about 5 wt % of the humectant, about 5 wt % citric acid, about 0.2 wt % calcium L-ascorbate, about 0.1 wt % of the non-naturally occurring preservative, about 1 wt % of the natural preservative, with the balance of the moisturizer composition being the water:glycerin mixture.

A sixteenth embodiment of the invention is a moisturizer composition according to the fifteenth embodiment, wherein the composition comprises about 83-84 wt % of the water:glycerin mixture.

A seventeenth embodiment of the invention is a moisturizer composition according to the fifteenth embodiment, wherein the composition further comprise about 0.5 to about 2 wt % of an essential oil.

A eighteenth embodiment of the invention is a moisturizer composition according to the seventeenth embodiment, wherein the composition comprises about 81-83 wt % of the water:glycerin mixture.

An nineteenth embodiment of the invention is a moisturizer composition according to any one of the fifteenth through eighteenth embodiments, wherein the water:glycerin mixture has a water:glycerin weight ratio of about 50:50.

A twentieth embodiment of the invention is a moisturizer composition according to any one of the fourteenth through nineteenth embodiments, wherein the skin barrier enhancer is potassium phosphate, the humectant is urea, the non-naturally occurring preservative is potassium sorbate, and the natural preservative is Naticide.

A twenty-first embodiment of the invention is a moisturizer composition according to any one of the first through twentieth embodiments, wherein the composition of free of emulsifiers.

A twenty-second embodiment of the invention is a moisturizer composition according to any one of the first through twentieth embodiments, wherein the composition of free of surfactants.

A twenty-third embodiment of the invention is a moisturizer composition according to any one of the first through twentieth embodiments, wherein the composition of free of thickening agents.

A twenty-fourth embodiment of the invention is a moisturizer composition according to any one of the first through twentieth embodiments, wherein the composition of free of rheology modifiers.

A twenty-fifth embodiment of the invention is a moisturizer composition according to any one of the first through twentieth embodiments, wherein the composition of free of two or more of emulsifiers, surfactants, thickening agents and rheology modifiers.

A twenty-sixth embodiment of the invention is a moisturizer composition according to any one of the first through twentieth embodiments, wherein the composition of free of emulsifiers, surfactants, thickening agents and rheology modifiers.

A twenty-seventh embodiment of the invention is a method of moisturizing a surface, the method comprising applying a moisturizer composition according to any one of the first through twenty-sixth embodiments onto a surface.

A twenty-eighth embodiment of the invention is a method according to the twenty-seventh embodiment, wherein the surface is skin of a mammal.

A twenty-ninth embodiment of the invention is a method according to the twenty-eighth embodiment, wherein the mammal is a human.

A thirtieth embodiment of the invention is a method according to the twenty-eighth embodiment, wherein the skin of a mammal is a leather.

A thirty-first embodiment of the invention is a method according to the twenty-seventh embodiment, wherein the surface is made of a plastic.

A thirty-second embodiment of the invention is a method according to the thirty-first embodiment, wherein the plastic is at least partially made of a material is selected from the group consisting of polypropylene (PP) plastics, acrylonitrile-butadiene-styrene (ABS) block copolymer plastics, styrene maleic anhydride (SMA) plastics, polyphenylene ether (PPE) plastics, polyvinyl chloride (PVC) plastics, polyurethane (PU) plastics, and any combination thereof.

### DETAILED DESCRIPTION

The following description of the embodiments is merely exemplary in nature and is in no way intended to limit the subject matter of the present disclosure, their application, or uses.

As used throughout, ranges are used as shorthand for describing each and every value that is within the range. Any value within the range can be selected as the terminus of the range. Unless otherwise specified, all percentages and amounts expressed herein and elsewhere in the specification should be understood to refer to percentages by weight.

For the purposes of this specification and appended claims, unless otherwise indicated, all numbers expressing quantities, percentages or proportions, and other numerical values used in the specification and claims, are to be understood as being modified in all instances by the term “about.” The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent, alternatively ±5 percent, and alternatively ±1 percent of the given numeric value provided such a deviation does not alter the end function or result of the value. Accordingly, unless indicated to the contrary, the numerical parameters set forth in this specification and attached claims are approximations that can vary depending upon the desired properties sought to be obtained by the present invention.

It is noted that, as used in this specification and the appended claims, the singular forms “a,” “an,” and “the,” include plural references unless expressly and unequivocally limited to one referent. As used herein, the term “include” and its grammatical variants are intended to be non-limiting, such that recitation of items in a list is not to the exclusion of other like items that can be substituted or added to the listed items. For example, as used in this specification and the following claims, the terms “comprise” (as well as forms, derivatives, or variations thereof, such as “comprising” and “comprises”), “include” (as well as forms, derivatives, or variations thereof, such as “including” and “includes”) and “has” (as well as forms, derivatives, or variations thereof, such as “having” and “have”) are inclusive (i.e., open-ended) and do not exclude additional elements or steps. Accordingly, these terms are intended to not only cover the recited element(s) or step(s), but may also include other elements or steps not expressly recited. Furthermore, as used herein, the use of the terms “a” or “an” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The term “topical application” or variations thereof, as used herein, means to apply or spread the compositions of the present invention onto the surface of the skin.

The term “pharmaceutically acceptable amount” or variations thereof, as used herein, means an amount of a compound, component, or composition sufficient to induce a desired positive benefit, but low enough to avoid serious side effects, i.e., to provide a reasonable benefit to risk ratio, within the scope of sound medical judgment.

In accordance with various aspect of the present disclosure, the present invention relates to moisturizing compositions. In some instances, the moisturizing compositions have been found very effective at moisturizing the skin of a mammalian when applied topically in a pharmaceutically acceptable amount. In some instances the moisturizing compositions have been found very effective at moisturizing inanimate surfaces and/or materials, such as certain fabrics, textiles or surfaces made from certain fabrics or textiles, that may be prone to drying, cracking, ultraviolet (UV) damage, or other similar types of damage over time.

In some instances, moisturizing compositions according to the invention are free of emulsifiers. In some instances, moisturizing compositions according to the invention are free of surfactants. In some instances, moisturizing compositions according to the invention are free of thickening agents. In some instances, moisturizing compositions according to the invention are free of rheology modifiers. In some instances, moisturizing compositions according to the invention are free of two or more of emulsifiers, surfactants, thickening agents and rheology modifiers. In some instances, moisturizing compositions according to the invention are free of emulsifiers, surfactants, thickening agents and rheology modifiers.

In accordance with various aspects of the disclosure, moisturizing compositions according to the invention are made of various compounds contained within a water-glycerin based carrier. Moisturizing compositions according to the invention are made of at least about 60 wt % and at most about 95 wt % of the water-glycerin based carrier (alternatively referred to herein as a “water:glycerin mixture”). In some instances, the moisturizing compositions are made of about 65 wt % to about 92.5 wt %, alternatively from about 70 wt % to about 90 wt %, alternatively from about 75 wt % to about 87.5 wt %, alternatively from about 80 wt % to about 85 wt %, and alternatively from about 81 wt % to about 84 wt % of the water-glycerin based carrier. The water-glycerin based carrier has a water-to-glycerin weight/weight % (w/w %) ratio ranging from about 45:55 to about 85:15, preferably from about 50:50 to about 80:20, and more preferably from about 50:50 to about 70:30. In some instances, the water-glycerin based carrier has a water-to-glycerin w/w % ratio of about 50:50. In some instances, the water-glycerin based carrier has a water-to-glycerin w/w % ratio of about 60:40. In some instances, the water-glycerin based carrier has a water-to-glycerin w/w % ratio of about 70:30. In some instances, the water-glycerin based carrier has a water-to-glycerin w/w % ratio of about 80:20.

In accordance with various aspects of the disclosure, moisturizing compositions according to the invention include a variety of B vitamins including niacinamide (Vitamin B3), pantothenic acid (Vitamin B5), and pyridoxine (Vitamin B6). Moisturizing compositions according to the invention can have from about 2 wt % to about 10 wt % B vitamins. In some instances, the moisturizing compositions have from about 2.5 to about 9 wt %, alternatively from about 3 to about 8 wt %, alternatively from about 3.5 to about 7 wt %, alternatively from about 4 to about 6 wt %, alternatively from about 4.5 to about 5.5 wt %, and alternatively about 5 wt % B vitamins. Moisturizing compositions according to the invention can have from about 1 wt % to about 5 wt % niacinamide. In some instances, the moisturizing compositions have from about 1.5 to about 4 wt %, alternatively from about 2 to about 3 wt %, alternatively from about 2.25 to about 2.75 wt %, and alternatively about 2.5 w % niacinamide. Moisturizing compositions according to the invention can have from about 0.5 to about 2 wt % pantothenic acid. In some instances, the moisturizing compositions have from about 0.75 to about 1.75 wt %, alternatively from about 1 to about 1.5 wt %, alternatively from about 1.1 to about 1.3 wt %, and alternatively about 1.2 wt % pantothenic acid. Moisturizing compositions according to the invention can have from about 0.5 to about 2 wt % pyridoxine. In some instances, the moisturizing compositions have from about 0.75 to about 1.75 wt %, alternatively from about 1 to about 1.5 wt %, alternatively from about 1.1 to about 1.3 wt %, and alternatively about 1.2 wt % pyridoxine.

In accordance with various aspects of the disclosure, moisturizing compositions according to the invention include a skin barrier enhancer. Suitable skin barrier enhancers include, but are not limited to, potassium phosphate, dipotassium phosphate, phosphoric acid, squalane, squalene, linoleic acid, jojoba oil, coconut oil, almond oil, argan oil, borage oil, rosehip oil, sunflower oil, soybean oil, oat oil, palm kernel oil, and ceramides. In some instances, the use of potassium phosphate as the skin barrier enhancer is preferred. Moisturizing compositions according to the invention can have from about 0.05 to about 1 wt % of the skin barrier enhancer. In some instances, the moisturizing compositions have from about 0.075 to about 0.8 wt %, alternatively from about 0.1 to about 0.6 wt %, alternatively from about 0.125 to about 0.4 wt %, alternatively from about 0.15 to about 0.3 wt %, alternatively from about 0.175 to about 0.25 wt %, and alternatively about 0.2 wt % of the skin barrier enhancer. In some instances, a combination of skin barrier enhancers may be used, where the combination of skin barrier enhancers in total is from about 0.05 to about 1 wt %, alternatively from about 0.075 to about 0.8 wt %, alternatively from about 0.1 to about 0.6 wt %, alternatively from about 0.125 to about 0.4 wt %, alternatively from about 0.15 to about 0.3 wt %, alternatively from about 0.175 to about 0.25 wt %, and alternatively about 0.2 wt % of the moisturizer composition.

In accordance with various aspects of the disclosure, moisturizing compositions according to the invention include a humectant. Suitable humectants include, but are not limited to, urea, sorbitol, mannitol, propylene glycol, beet root extract, Aloe barbadensis leaf extract, Aloe andongensis leaf juice, Aloe andongensis extract, Albatrellus conjluens extract, aluminum dihydroxy allantoinate (allantoin), Averrhoa carambola leaf extract, Backhousia anisata leaf extract, Banksia spinulosa flower extract, Bidens pilosa extract, Ceramium rubrum extract, Citrus auratium Tachibana peel extract, Cnidium monnieri fruit extract, Cocos nucifera fruit juice, Coleus scutellarioides extract, Convolvulus arvensis extract, Dunaliella bardawill extract, Echium lycosis root extract, Entada phaseoloides leaf extract, Erica canaliculate extract, fig fruit extract, Ganoderma atrum extract, Ganoderma neo-japonicum extract, Gentiana prostrate extract, licorice root extract, Legenaria siceraria fruit extract, Lantana camara root extract, Larix europaea wood extract, Lepidium meyenii root extract, Lespedeza bicolor bark extract, Lycopodium clavatum extract, Macrotomia euchroma root extract, Magnolia kobus bark extract, Momordica charantia fruit extract, Momordica grosvenori fruit juice, Paulownia tomentosa leaf extract, Phalaenopsis amabillis extract, Phaseolus trilobus seed extract, Portulaca grandiflora extract, Portulaca oleracea extract, Primu glycolicla sikkimensis flower extract, Prostanthera incise leaf extract, Prunus persica bud extract, Pueraria lobata root extract, Ribes nigrum seed extract, Rosa rugosa leaf extract, Saussurea involucrata extract, Scutellaria baicalensis root extract, Sesamum indicum sprout extract, jojoba leaf extract, honey extract, albumen extract, algae oligosaccharides, agarose, lactose, jellyfish extract, fructose, fructan, diglycerin, glyceryl triacetate (triacetin), glucose, acetamide MEA, acetyl histidine, anserine, calcium fructoborate, cellulose succinate, choline chloride, erythritol, ethyl glucoside, 1,3-butylene glycol, hexylene glycol, hydrolyzed corn starch, hydroxylated jojoba oil, lactic acid, sodium lactate, lysine PCA, polyglucuronic acid, sodium PCA, maltose, mannose, maltitol, melibiose, dimethyl imidazolidinone, sodium hyaluronate, hyaluronic acid, salicylic acid, glycolic acid, phytic acid, gluconolactone, xylitol, and xylose. In some instances, the use of urea as the humectant is preferred. Moisturizing compositions according to the invention can have from about 1 to about 10 wt % of the humectant. In some instances, the moisturizing compositions have from about 2 to about 9 wt %, alternatively from about 3 to about 8 wt %, alternatively from about 4 to about 6 wt %, alternatively from about 4.5 to about 5.5 wt %, and alternatively about 5 wt % of the humectant. In some instances, a combination of humectants may be used, where the combination humectants in total is from about 1 to about 10 wt %, alternatively from about 2 to about 9 wt %, alternatively from about 3 to about 8 wt %, alternatively from about 4 to about 6 wt %, alternatively from about 4.5 to about 5.5 wt %, and alternatively about 5 wt % of the moisturizer composition.

In accordance with various aspects of the disclosure, moisturizing compositions according to the invention include citric acid. Moisturizing compositions according to the invention can have from about 1 to about 10 wt % citric acid. In some instances, the moisturizing compositions have from about 2 to about 9 wt %, alternatively from about 3 to about 8 wt %, alternatively from about 4 to about 6 wt %, alternatively from about 4.5 to about 5.5 wt %, and alternatively about 5 wt % citric acid.

In accordance with various aspects of the disclosure, moisturizing compositions according to the invention include an ascorbic acid salt or an ascorbic acid derivative. Suitable ascorbic acid salts and ascorbic acid derivatives include, but are not limited to, sodium L-ascorbate, calcium L-ascorbate, magnesium L-ascorbate, ascorbyl 6-palmitate, tetra-isopalmitoyl ascorbate, magnesium ascorbyl phosphate, sodium ascorbyl phosphate, ascorbyl 2-glucoside, ascorbyl 2-phosphate-6-palmitate, and 3-O-ethyl ascorbate. In some instances, the use of an ascorbic acid salt is preferred. In some instances, the use of calcium L-ascorbate as the ascorbic acid salt is preferred. Moisturizing compositions according to the invention can have from about 0.1 to about 0.5 wt % of the humectant. In some instances, the moisturizing compositions have from about 0.125 to about 0.4 wt %, alternatively from about 0.15 to about 0.3 wt %, alternatively from about 0.175 to about 0.25 wt %, alternatively from about 0.175 to about 0.225 wt %, and alternatively about 0.2 wt % of the ascorbic acid salt or ascorbic acid derivative. In some instances, a combination of ascorbic acid salts and/or ascorbic acid derivatives may be used, where the combination ascorbic acid salts and/or ascorbic acid derivatives in total is from about 0.1 to about 0.5 wt %, alternatively from about 0.125 to about 0.4 wt %, alternatively from about 0.15 to about 0.3 wt %, alternatively from about 0.175 to about 0.25 wt %, alternatively from about 0.175 to about 0.225 wt %, and alternatively about 0.2 wt % of the moisturizer composition.

In accordance with various aspects of the disclosure, moisturizing compositions according to the invention include a non-naturally occurring preservative (sometime referred to as a synthetic or chemical preservative). Suitable non-naturally occurring preservatives include, but are not limited to, potassium sorbate, triclosan, methylisothiazolinone, chlorphenesin, chloroxylenol, iodopropynyl butyl carbamate, methyldibromo glutaronitrile, formaldehyde, benzylhemiformal, diasolidinyl urea, imidazolidinyl urea, 2-bromo-2-nitropropane-1,3-diol, 1,3-bis(hydroxymethyl)-5,5-dimethylimidazolidine-2,4-dione, 1-(hydroxymethyl)-5,5-dimethylhydantoin, 1-(3-chloroallyl)-3,5,7-triaza-1-azoniaadamantane chloride, Sodium hydroxymethyl glycinate, phenoxyethanol, 2-butoxyethanol, 2-(2-butoxyethoxy)-ethanol, 2-(2-ethoxy)-ethanol, methylparaben, ethylparaben, propylparaben, butylparaben, isobutylparaben, 1,2-hexanediol, caprylyl glycol, caprylyl glyceryl ether, dehydroacetic acid, caprylhydroxamic acid, ethylhexylglycerin, glyceryl undecylenate, glycerol, and piroctone olamine. In some instances, the use of potassium sorbate as the non-naturally occurring preservative is preferred. Moisturizing compositions according to the invention can have from about 0.01 to about 0.2 wt % of the non-naturally occurring preservative. In some instances, the moisturizing compositions have from about 0.025 to about 0.175 wt %, alternatively from about 0.05 to about 0.15 wt %, alternatively from about 0.075 to about 1.25 wt %, alternatively from about 0.09 to about 1.1 wt %, and alternatively about 0.1 wt % of the non-naturally occurring preservative. In some instances, a combination of non-naturally occurring preservatives may be used, where the combination non-naturally occurring preservatives in total is from about 0.01 to about 0.2 wt %, alternatively from about 0.025 to about 0.175 wt %, alternatively from about 0.05 to about 0.15 wt %, alternatively from about 0.075 to about 0.125 wt %, alternatively from about 0.09 to about 0.11 wt %, and alternatively about 0.1 wt % of the moisturizer composition.

In accordance with various aspects of the disclosure, moisturizing compositions according to the invention include a natural preservative. Suitable natural preservatives include, but are not limited to, benzoic acid, benzyl alcohol, sodium benzoate, potassium benzoate, sorbic acid, salicylic acid, p-ani sic acid, Lactobacillus ferment, Lactobacillus, Cocos Nucifera (coconut) fruit extract, Japanese honeysuckle extract, Leuconostoc/radish root ferment filtrate, Populus Tremuloides bark extract, Salix Nigra (willow) bark extract, Leuconostoc ferment filtrate, Sambucus Nigra fruit extract, Musa Sapientum (banana) fruit extract, sodium levulinate, sodium anisate, glycerin, citrus reticulata fruit extract, citrus aurantium amara fruit extract, Citrus sinensis peel extract, ascorbic acid, citric acid, lactic acid, D-glucono-1,5-lactone, D-gluconic acid calcium salt, pentylene glycol, and phenethyl alcohol. In some instances, the natural preservative may be provided from a commercially available composition. In some instances the commercially available composition is Naticide® (Sinerga SPA, Italy), which is a vegetable derived ingredient with a wide spectrum of activity, being effective against Gram+, Gram-, yeasts and molds in a pH range between 4-9, and having a vanilla-like scent. In some instances, the commercially available composition is Dermasoft® 1388 ECO (Evonik Dr. Straetmans GmbH, Hamburg, Germany), which is a mixture having a pH range of 4.0-5.5 and comprising glycerin, water sodium levulinate and sodium anisate. In some instances, the commercially available composition is Leucidal® Liquid (Lotioncrafter, Eastsound, Wash., USA) which is a mixture of 48-52% water and 48-52% Leuconostoc/radish root ferment filtrate having a pH range of 4.0-6.0. In some instances, the commercially available composition is Biosecur® Organic Personal Care C160S (Sharon Laboratories, Israel), which is a mixture comprising glycerin, citrus reticulata fruit extract, citrus aurantium amara fruit extract, Citrus sinensis peel extract, ascorbic acid, citric acid, lactic acid, and water. In some instances, the commercially available composition is Geogard Ultra™ (Lonza Ltd., Basel, Switzerland) which is a mixture of 7.7-8.3% dehydroacetic acid, 85-89% benzyl alcohol, and 4% water. In some instances, the commercially available composition is Geogard® ECT (Lonza Ltd., Basel, Switzerland) which is a mixture of 77-86% benzyl alcohol, 8-15% salicylic acid, 3-5% glycerin and 1-4% sorbic acid. In some instances, the use of Naticide as the natural preservative is preferred. Moisturizing compositions according to the invention can have from about 0.5 to about 2 wt % of the natural preservative. In some instances, the moisturizing compositions have from about 0.6 to about 1.75 wt %, alternatively from about 0.7 to about 1.5 wt %, alternatively from about 0.8 to about 1.25 wt %, alternatively from about 0.9 to about 1.1 wt %, and alternatively about 1 wt % of the natural preservative. In some instances, a combination of natural preservatives may be used, where the combination natural preservatives in total is from about 0.01 to about 0.2 wt %, alternatively from about 0.025 to about 0.175 wt %, alternatively from about 0.05 to about 0.15 wt %, alternatively from about 0.075 to about 1.25 wt %, alternatively from about 0.09 to about 1.1 wt %, and alternatively about 0.1 wt % of the moisturizer composition.

In accordance with various aspects of the disclosure, moisturizing compositions according to the invention include an essential oil. In some instances, essential oils may provide the moisturizing compositions with a desirable smell or fragrance. In some instances, essential oils, in addition to providing a desirable smell or fragrance, may provide the moisturizing compositions with a property beneficial to the user such as, but limited to, skin hydration or moisturization, inhibition of skin pigmentation, skin abrasion healing, skin burn treatment, rash treatment, bug bite treatment, eczema treatment, acne treatment, treatment of bacterial or fungal infections, wart treatment, and anti-inflammatory treatments. Suitable natural preservatives include, but are not limited to, alfalfa oil, almond oil, arborvitae oil, argan oil, avocado oil, babassu oil, basil oil, bergamot oil, camellia seed oil, canola oil, carrot seed oil, cassia oil, castor oil, cedarwood oil, cinnamon oil, clary sage oil, clove oil, coconut oil, copaiba oil, eucalyptus oil, frankincense oil, geranium oil, grapefruit oil, grapeseed oil, helichrysum oil, hyssop oil, jojoba oil, lavender oil, lemon oil, lemongrass oil, manila oil, moringa oil, myrrh oil, neroli oil, olive oil, orange oil, oregano oil, palmarosa oil, patchouli oil, peppermint oil, pine oil, pomegranate oil, roman chamomile oil, rice bran oil, rose oil, rosehip oil, rosemary oil, sandalwood oil, spearmint oil, sunflower oil, sweet orange oil, tangerine oil, tea tree oil, thyme oil, wintergreen oil, vetiver oil, and ylang oil. Moisturizing compositions according to the invention can have from about 0.5 to about 2 wt % of the essential oil. In some instances, a combination of essentials may be used. When a combination of essential oils is used, it is preferred that the combined amount of essential oils total about 0.5 to about 2.0 wt % of the moisturizer composition.

In accordance with various aspects of the disclosure, moisturizing compositions according to the invention can be applied onto a surface to moisturize said surface. In some instances, the surface is the skin of mammal and the amount of moisturizing composition applied onto the skin is a pharmaceutically acceptable amount. In some instances, the surface is human skin. In some instances, the surface is a leather. In some instances, the surface is a plastic. In some instances, the plastic is in the form of a woven or non-woven textile or fabric. In some instances, the plastic is at least partially made of a material such as, but not limited to polypropylene (PP) plastics, acrylonitrile-butadiene-styrene (ABS) block copolymer plastics, styrene maleic anhydride (SMA) plastics, polyphenylene ether (PPE) plastics, polyvinyl chloride (PVC) plastics, polyurethane (PU) plastics, and any combination thereof.

### Examples

Example 1—Preparation of Moisturizer Composition. Water (40 ml) and a stir bar were placed in a large beaker that was situated on a stir plate. Potassium sorbate, in an amount sufficient to provide the final moisturizer composition with 0.1 wt % potassium sorbate, was added and stirred under ambient conditions until dissolved. Pantothenic acid, in an amount sufficient to provide the final moisturizer composition with 1.2 wt % potassium sorbate, was then added and stirred under ambient conditions until dissolved. Niacinamide, pyroxidine, potassium phosphate, urea, citric acid and calcium L-ascorbate (in amounts sufficient to provide the final moisturizer composition with 2.5 wt % niacinamide, 1.2 wt % pyroxidine, 0.2 wt % potassium phosphate, 5 wt % urea, 5 wt % citric acid and 0.2 wt % calcium L-ascorbate) were then added and stirred under ambient conditions until dissolved. Naticide® in an amount sufficient to provide the final moisturizer composition with 1 wt % Naticide® was then added and stirred under ambient conditions until dissolved. Finally, glycerin was added while stirring under ambient conditions until the final volume of the mixture was 100 ml. After addition of the glycerin was complete, the final moisturizer composition was obtained.

Example 2—Stability Testing. In this example, the moisturizer composition of Example 1 was subjected to stability testing. Stability tests were carried out at temperatures of 4±2° C., 25±2° C. and 40±2° C. for three months. During this period and at each of these temperatures, the composition was monitored at 5 endpoints: T=0, T=15 days, T=30 days, T=60 days and T=90 days. The stability tests included a centrifugation test, a density test, a viscosity test, a pH test, and assessment of organoleptic properties (aspect and odor).

The centrifugation test is intended to observe out any phase separation or solid precipitation exhibited by a sample. Samples were centrifuged at 3340 rpm/min for 2×15 min with Fisher Scientific Centrific Model 228. The results were expressed as Stable/Separated/Oil on the Top/Bubbles. After making comparison with TO, the results were concluded as stable (S) or not stable (NS). In the density test, sample density was measured with Pycnometer. The results are expressed as “value”±0.001 g/ml. In the viscosity test, sample viscosity was measured with Brookfield Dial Viscometer Model RVF. The results are expressed as “value”±0.2% cP (or N/A: not applicable). In the pH test, pH was measured with pH-meter Milwaukee MW102 pH/Temp Meter at the temperature of 25° C. The results are expressed as “value”±0.01 pH unit. In the organoleptic properties assessment, sample appearance, color and odor were determined by visual and olfactory inspection, and they were evaluated by making comparison with TO. The results are expressed as compliant (C) or not compliant (NC). The results of these tests are provided below in Tables 1-2.

TABLE 1
Study Parameter

| | Centrifugation Test | | | Density | | | pH | | |
|---|---|---|---|---|---|---|---|---|---|
| Temp. | 4° C. | 25° C. | 40° C. | 4° C. | 25° C. | 40° C. | 4° C. | 25° C. | 40° C. |
| T0 | — | S | — | — | 1.169 | — | — | 3.04 | — |
| T15 | S | S | S | 1.171 | 1.191 | 1.170 | 3.03 | 3.01 | 3.21 |
| T30 | S | S | S | 1.173 | 1.197 | 1.171 | 3.15 | 3.15 | 3.30 |
| T60 | S | S | S | 1.169 | 1.169 | 1.168 | 3.32 | 3.35 | 3.80 |
| T90 | S | S | S | 1.170 | 1.170 | 1.171 | 3.23 | 3.33 | 3.98 |

TABLE 2
Study Parameter

| | Viscosity | | | Odor | | | Appearance | | |
|---|---|---|---|---|---|---|---|---|---|
| Temp. | 4° C. | 25° C. | 40° C. | 4° C. | 25° C. | 40° C. | 4° C. | 25° C. | 40° C. |
| T0 | — | N/A | — | — | C | — | — | C | — |
| T15 | N/A | N/A | N/A | C | C | C | C | C | C |
| T30 | N/A | N/A | N/A | C | C | C | C | C | C |
| T60 | N/A | N/A | N/A | C | C | C | C | C | C |
| T90 | N/A | N/A | N/A | C | C | C | C | C | C |

Example 3—Challenge Testing. In this example, the moisturizer composition of Example 1 was subjected to challenge testing. A challenge test is a predictive method useful to evaluate the effectiveness of a preserving system used in the formulation of, for example, topical pharmaceuticals and cosmetics. In the challenge tests, we overdo the example 1 moisturizer composition was inoculated with very high concentrations various microbial strains, and their reduction in growth is evaluated at different end times. The challenge tests were conducted in accordance with that described in U.S. Pharmacopoeia (USP) 61. The test was preceded by an examination of the total microbial load of the product.

The inoculum was carried out with various microbial strains at different concentrations, as reported in Table 3.

TABLE 3

| | | Sample Inocule |
|---|---|---|
| | | Concentration |
| Microbial Strain | Growth Medium | (CFU/g) |
| Escherichia coli | Casein soya bean digest agar | 5.1 × 10⁵ |
| ATCC 8739 | | |
| Pseudomonas aeruginosa | Casein soya bean digest agar | 6.2 × 10⁵ |
| ATCC 9027 | | |
| Staphylococcus aureus | Casein soya bean digest agar | 9.1 × 10⁵ |
| ATCC 6538 | | |
| Candida albicans | Sabouraud-dextrose agar | 1.8 × 10⁵ |
| ATCC 10231 | | |
| Aspergillus brasiliensis | Sabouraud-dextrose agar | 1.3 × 10⁵ |
| ATCC 16404 | | |

The inoculum was prepared by cultivating the bacteria on casein soya bean digest agar medium and Sabouraud glucose agar for fungi. Bacteria were incubated at 32.5±2.5° C. for 18-24 hours, Candida at 22.5±2.5° C. for 44-52 hours and the Aspergillus at 22.5±2.5° C. for 6-10 days. The different microbial strains were suspended in a physiologic solution and inoculated in the tested product at a final concentration ranging between 10⁵-10⁶ for all the strains.

The treated samples were then stored at room temperature protected from light until when plated for the microbial count. The concentration of viable cells at every end-time were determined by the plate count method, diluting the product in a solution of sodium chloride-peptone added with neutralizers for the most common preservatives (polysorbate 80, soya lecithin, thiosulfate, L-Histidine). The microbial count at different endpoints was carried out by diluting 1 g/ml of product up to 1×10⁶ times and plating each dilution in a petri dish with selective agar medium. The plates were kept at 32.5±2.5° C. (bacteria) or at 22.5±2.5° C. (yeast and mold) for the time necessary for a good growth (3-5 days for bacteria and yeast, 3-7 days for molds). The U.F.C. (Unity Forming Colony) value for each gram or milliliter of product was obtained from the number of colonies on the plate for the dilution factor. To evaluate the microbial reduction in time, plate counts were carried out at three end-times normally after 7, 14 and 28 days from the starting inoculum.

For effectiveness against bacterial growth, the moisturizing composition of Example 1 was considered effective if it exhibited an NTL 2.0 log reduction from the initial bacterial count at 14 days, and no increase (i.e., not more the 0.5 log 10 unit) from the 14 days count at 28 days. For effectiveness against yeast and molds growth, the moisturizing composition of Example 1 was considered effective if it exhibited no increase (i.e., not more the 0.5 log 10 unit) in yeast or molds growth from the initial calculated count after 14 days and 28 days. The total microbial count for each tested strain after 7, 14 and 28 days is summarized below in Table 4, and the antimicrobial growth reduction efficacy is summarized below in Table 5.

TABLE 4

| Strain | E. coli | P. aeruginosa | S. aureus | C. albicans | A. brasiliensis |
|---|---|---|---|---|---|
| CFU Inoculum | 5.1 × 10⁵ | 6.2 × 10⁵ | 9.1 × 10⁵ | 1.8 × 10⁵ | 1.3 × 10⁵ |
| CFU 7 days | <10 | <10 | <10 | <10 | 50 |
| Microbial | >4.71 | >4.79 | >4.96 | >4.26 | 3.41 |
| reduction (log) | | | | | |
| Reduction | >99.99 | >99.99 | >99.99 | >99.99 | 99.96 |
| effectiveness | | | | | |
| (%) | | | | | |
| CFU 14 days | <10 | <10 | <10 | <10 | <10 |
| Microbial | >4.71 | >4.79 | >4.96 | >4.26 | >4.11 |
| reduction (log) | | | | | |
| Reduction | >99.99 | >99.99 | >99.99 | >99.99 | >99.99 |
| effectiveness | | | | | |
| (%) | | | | | |
| CFU 28 days | <10 | <10 | <10 | <10 | <10 |
| Microbial | >4.71 | >4.79 | >4.96 | >4.26 | >4.11 |
| reduction (log) | | | | | |
| Reduction | >99.99 | >99.99 | >99.99 | >99.99 | >99.99 |
| effectiveness | | | | | |
| (%) | | | | | |

TABLE 5

| Time | E. coli | P. aeruginosa | S. aureus | C. albicans | A. brasiliensis |
|---|---|---|---|---|---|
| 14 days | >2log | >2log | >2log | * | * |
| 28 days | * | * | * | * | * |

* growth not more than 0.5log 10 unit

Based on the data shown in Tables 4-5, it is clear that the moisturizing composition of Example 1 is effective at preventing the growth of each microbial strain tested and satisfies the requirements for the preservation efficacy test for topically used products according to the USP 61 challenge test.

What is claimed is:

1. A moisturizer composition, the composition comprising:
about 1 to about 5 wt % niacinamide;
about 0.5 to about 2 wt % pantothenic acid;
about 0.5 to about 2 wt % pyridoxine;
about 0.05 to about 1 wt % of a skin barrier enhancer;
about 1 to about 10 wt % of a humectant;
about 1 to about 10 wt % citric acid;
about 0.1 to about 0.5 wt % an ascorbic acid salt or an ascorbic acid derivative;
about 0.01 to about 0.2 wt % of a non-naturally occurring preservative;
about 0.5 to about 2 wt % of a natural preservative; and
balance of a water:glycerin mixture, the water:glycerin mixture having at least about 50 wt % water.

2. The moisturizer composition of claim 1, wherein the water:glycerin mixture has a water:glycerin weight ratio ranging from about 50:50 to about 80:20.

3. The moisturizer composition of claim 1, wherein the water:glycerin mixture has a water:glycerin weight ratio ranging from about 50:50 to about 70:30.

4. The moisturizer composition of claim 1, wherein the skin barrier enhancer is selected from the group consisting of potassium phosphate, dipotassium phosphate, phosphoric acid, squalane, squalene, linoleic acid, jojoba oil, coconut oil, almond oil, argan oil, borage oil, rosehip oil, sunflower oil, soybean oil, oat oil, palm kernel oil, and ceramides.

5. The moisturizer composition of claim 1, wherein the humectant is selected from the group consisting of urea, sorbitol, mannitol, propylene glycol, beet root extract, Aloe barbadensis leaf extract, Aloe andongensis leaf juice, Aloe andongensis extract, Albatrellus conjluens extract, aluminum dihydroxy allantoinate (allantoin), Averrhoa carambola leaf extract, Backhousia anisata leaf extract, Banksia spinulosa flower extract, Bidens pilosa extract, Ceramium rubrum extract, Citrus auratium Tachibana peel extract, Cnidium monnieri fruit extract, Cocos nucifera fruit juice, Coleus scutellarioides extract, Convolvulus arvensis extract, Dunaliella bardawill extract, Echium lycosis root extract, Entada phaseoloides leaf extract, Erica canaliculate extract, fig fruit extract, Ganoderma atrum extract, Ganoderma neo-japonicum extract, Gentiana prostrate extract, licorice root extract, Legenaria siceraria fruit extract, Lantana camara root extract, Larix europaea wood extract, Lepidium meyenii root extract, Lespedeza bicolor bark extract, Lycopodium clavatum extract, Macrotomia euchroma root extract, Magnolia kobus bark extract, Momordica charantia fruit extract, Momordica grosvenori fruit juice, Paulownia tomentosa leaf extract, Phalaenopsis amabillis extract, Phaseolus trilobus seed extract, Portulaca grandiflora extract, Portulaca oleracea extract, Primuglycolicla sikkimensis flower extract, Prostanthera incise leaf extract, Prunus persica bud extract, Pueraria lobata root extract, Ribes nigrum seed extract, Rosa rugosa leaf extract, Saussurea involucrata extract, Scutellaria baicalensis root extract, Sesamum indicum sprout extract, jojoba leaf extract, honey extract, albumen extract, algae oligosaccharides, agarose, lactose, jellyfish extract, fructose, fructan, diglycerin, glyceryl triacetate (triacetin), glucose, acetamide MEA, acetyl histidine, anserine, calcium fructoborate, cellulose succinate, choline chloride, erythritol, ethyl glucoside, 1,3-butylene glycol, hexylene glycol, hydrolyzed corn starch, hydroxylated jojoba oil, lactic acid, sodium lactate, lysine PCA, polyglucuronic acid, sodium PCA, maltose, mannose, maltitol, melibiose, dimethyl imidazolidinone, sodium hyaluronate, hyaluronic acid, salicylic acid, glycolic acid, phytic acid, gluconolactone, xylitol, xylose, and any combination thereof.

6. The moisturizer composition of claim 1, wherein the humectant is urea.

7. The composition of claim 1, wherein the ascorbic acid salt is calcium L-ascorbate.

8. The composition of claim 1, wherein the non-naturally occurring preservative is selected from the group consisting of potassium sorbate, triclosan, methylisothiazolinone, chlorphenesin, chloroxylenol, iodopropynyl butyl carbamate, methyldibromo glutaronitrile, formaldehyde, benzylhemiformal, diasolidinyl urea, imidazolidinyl urea, 2-bromo-2-nitropropane-1,3-diol, 1,3-bis(hydroxymethyl)-5,5-dimethylimidazolidine-2,4-dione, 1-(hydroxymethyl)-5,5-dimethylhydantoin, 1-(3-chloroallyl)-3,5,7-triaza-1-azoniaadamantane chloride, Sodium hydroxymethyl glycinate, phenoxyethanol, 2-butoxyethanol, 2-(2-butoxyethoxy)-ethanol, 2-(2-ethoxy)-ethanol, methylparaben, ethylparaben, propylparaben, butylparaben, isobutylparaben, 1,2-hexanediol, caprylyl glycol, caprylyl glyceryl ether, dehydroacetic acid, caprylhydroxamic acid, ethylhexylglycerin, glyceryl undecylenate, glycerol, piroctone olamine, and any combination thereof.

9. The composition of any one of claim 1, wherein the non-naturally occurring preservative is potassium sorbate.

10. The composition of claim 1, wherein the natural preservative is selected from the group consisting of benzoic acid, benzyl alcohol, sodium benzoate, potassium benzoate, sorbic acid, salicylic acid, p-anisic acid, Lactobacillus ferment, Lactobacillus, Cocos Nucifera (coconut) fruit extract, Japanese honeysuckle extract, Leuconostoc/radish root ferment filtrate, Populus Tremuloides bark extract, Salix Nigra (willow) bark extract, Leuconostoc ferment filtrate, Sambucus Nigra fruit extract, Musa Sapientum (banana) fruit extract, sodium levulinate, sodium anisate, glycerin, citrus reticulata fruit extract, citrus aurantium amara fruit extract, Citrus sinensis peel extract, ascorbic acid, citric acid, lactic acid, D-glucono-1,5-lactone, D-gluconic acid calcium salt, pentylene glycol, phenethyl alcohol, Naticide and any combination thereof.

11. The composition of claim 1, wherein the natural preservative is Naticide.

12. The moisturizer composition of claim 1, further comprising about 0.5 to about 2 wt % of an essential oil.

13. The moisturizer composition of claim 1, wherein the composition comprises:
about 2.5 wt % niacinamide;
about 1.2 wt % pantothenic acid;
about 1.2 wt % pyridoxine;
about 0.2 wt % of the skin barrier enhancer;
about 5 wt % of the humectant;
about 5 wt % citric acid;
about 0.2 wt % calcium L-ascorbate;
about 0.1 wt % of the non-naturally occurring preservative;
about 1 wt % of the natural preservative; and
balance of the water:glycerin mixture.

14. The moisturizer composition of claim 1, wherein the moisturizer composition of free of two or more of emulsifiers, surfactants, thickening agents and rheology modifiers.

15. The moisturizer composition of claim 1, wherein the moisturizer composition of free of emulsifiers, surfactants, thickening agents and rheology modifiers.

16. A method of moisturizing a surface, the method comprising applying a moisturizer composition according to claim 1 onto a surface.

17. The method of claim 16, wherein the surface is skin of a mammal.

18. The method of claim 17, wherein the skin of a mammal is a leather.

19. The method of claim 16, the surface is made of a plastic.

20. The method of claim 19, wherein the plastic is at least partially made of a material selected from the group consisting of polypropylene (PP) plastics, acrylonitrile-butadiene-styrene (ABS) block copolymer plastics, styrene maleic anhydride (SMA) plastics, polyphenylene ether (PPE) plastics, polyvinyl chloride (PVC) plastics, polyurethane (PU) plastics, and any combination thereof.
